(19) United States
(12) Patent Application Publication
(10) Pub. No.: US 2011/0189222 A1
(43) Pub. Date: Aug. 4, 2011

(54) COMPOSITION FOR WOUND HEALING

(76) Inventors: Suhaila Mohamed, Serdang (MY); Rosalina Roslan Tan, Serdang (MY); Samaneh Ghasemi, Serdang (MY); Mehdi Javadi, Serdang (MY); Maslia Manja, Serdang (MY); Kharidah Muhammad, Serdang (MY)

(21) Appl. No.: 13/121,580

(22) PCT Filed: Sep. 29, 2009
(86) PCT No.: PCT/MY09/00162

§ 371 Date: Apr. 26, 2011

(30) Foreign Application Priority Data
Sep. 30, 2008 (MY) PI 20083894

(51) Int. Cl. A61K 36/02; A61K 36/39; A61K 36/889; A61P 17/02

(52) U.S. Cl. 424/195.17; 424/725; 424/727

(57) ABSTRACT

A composition to be orally administered and/or topically applied for enhancing wound healing, tissue repair and cardiovascular health protection comprising an extract of vegetative parts of Ipomoea, Arecaceae plant family and/or seaweeds through extraction using alcoholic solvent with or without combination with a co-solvent, which may be aqueous or organic as an active ingredient.

### FIELD OF INVENTION

The present invention relates to a composition capable of enhancing wound healing, tissue repair and cardiovascular health protection. In more specific, the disclosed composition can accelerate the healing process which is especially useful for patients with compromised healing process such as diabetic patients.

### BACKGROUND OF THE INVENTION

Acute wounds heal in an orderly progression, through artificially defined phases of coagulation, inflammation, matrix synthesis and deposition, angiogenesis, fibroplasia, epithelialization, contraction, and remodeling. Generally, the healing process can be simplified as inflammation, fibroplasia, and maturation. The end result of uncomplicated healing is a fine scar with little fibrosis, minimal if any wound contraction, and a return to near normal tissue architecture and organ function. On the other hand, if a wound does not heal in an orderly or timely sequence, or if the healing process does not result in structural integrity, then the wound is considered chronic. Skin ulcers are probably the most common types of chronic wounds. These wounds can be created or perpetuated by many factors, including vascular insufficiency, either venous or arterial, prolonged inflammation, pressure necrosis, physical agents, infection, and cancer. Seventy percent of skin wounds, however, are due to pressure ulcers, diabetic foot ulcers, and venous ulcers. Normally, antibiotics like mupirocin, metranidazole, polymyxin B, Neosporin, or bacitracin are applied to the wounded area to avoid bacterial infestation that may further deteriorate the condition if happened. However, such practice may able to rid off possible bacterial infestation but not necessary lead to healing of the wound. Moreover, these chemically synthesized drugs tend to cause tolerance or side effect onto the users. Chronic wounds and their treatment are a huge burden on the healthcare system, both in terms of cost, time and attention of care required. The lost in productivity and decreased quality of life is immeasurable. Therefore, it is desired to have non-chemically synthesized medicament which can promote the healing process in these patients thus relieving their agony.

Japan patent publication no. 2007075425 provides a wound covering material. In their application the wound covering material contains alginic acids, preferably in the form of sodium alginate, which can be of origin form the plants of genus Lessonia, Laminaria group, or Ascophyllum.

In worldwide patent no. WO2004103280 discloses a composition for treatment of Type II diabetes mellitus comprising an amount of seaweed extract and a nitrogen donor in an effective amount. Preferably, the algae used for extraction derives from brown, red and green algae which the extract is then sulfinated to carry out the therapeutic effect.

Daniels filed an United State patent with publication no. 2007082868 claiming a method for treating Type II diabetes mellitus via administrating to a mammal an amount of seaweed extract in an effective doses.

### SUMMARY OF THE INVENTION

The present invention aims to provide a composition to enhance or promote wound healing. In more specific, the disclosed composition is taken orally or applied topically for speeding up healing of wounded skin or other tissues.

Further object is to provide a plant-based composition promoting wound healing yet may provide no adverse effect due to long term use of the composition.

Still another object of the present invention is to provide a method to acquire the disclosed extract with wound healing promoting effect.

At least one of the preceding objects is met, in whole or in part, by the present invention, in which one of the embodiment of the present invention involves a composition to be orally administered and/or topically applied for enhancing wound healing comprising an extract of vegetative parts (especially foliages) of plant family of Arecaceae or Ipomoea, optionally with seaweeds extract as well through extraction using alcoholic solvent with or without combination with a co-solvent, which may be aqueous or organic, as an active ingredient.

In further embodiment, the plant part of the Ipomoea or Arecaceae plant family is foliages. In subsequent embodiment, the Ipomoea species is preferably Ipomoea batatas, while the species of Arecaceae plant family is preferably but not limited to Elaeis guineensis, Elaeis oleifera, Phoenix dactylifera and Cocos nucifera. Optionally extraction of the seaweeds may come from any one or combination of Eucheuma cottonii, Kappaphycus alvarezii, Caulerpa lentillifera and Sargassum polycystum.

In order to extract the desired metabolites of the plants to carry out the therapeutic effect, the alcoholic solvent with or without combination with a co-solvent, which may be aqueous or organic is any one or combination of alcohol, water, acetone, acetonitrile, chloroform, dichloromethane and petroleum ether.

### BRIEF DESCRIPTION OF THE DRAWINGS

FIG. 1 shows the wound healing effect on the tested normal Sprague Drawley rats at different time interval of the experiments using composition prepared through different method and materials; and

FIG. 2 shows the wound healing effect on to the tested diabetic rat groups at different time interval of the experiments using composition prepared through different method and materials;

FIG. 3 is a graph showing efficiency of different extracts and negative controls applied on normal rats after 12 days administration;

FIG. 4 is a graph showing efficiency of different extracts and negative controls applied on diabetic rats after 12 days administration;

FIG. 5 shows the histology study on the wound on normal rats after 12 days administration of (a) water, (b) honey, (c) sweet potato and (d) Seaweed and palm leaves;

FIG. 6 shows that all groups of normal rats treated with the extracts displayed reduced MDA activity;

FIG. 7 shows that the groups of normal rats treated with seaweed and sweet potato extracts displayed increased catalase activity.

FIG. 8 shows the histology study on the liver of (a) untreated diabetic rats; (b) diabetic rats treated with seaweed extract; and (c) diabetic rats treated with palm extract; and

FIG. 9 shows the histology study on the kidney of (a) untreated diabetic rats; (b) diabetic rats treated with seaweed extract; and (c) diabetic rats treated with palm extract.

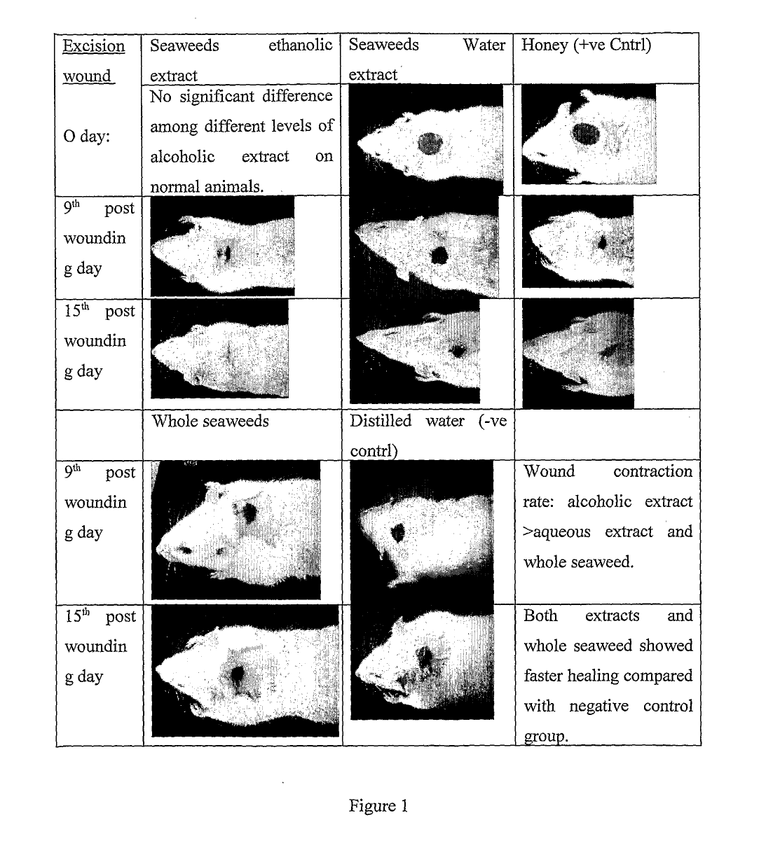

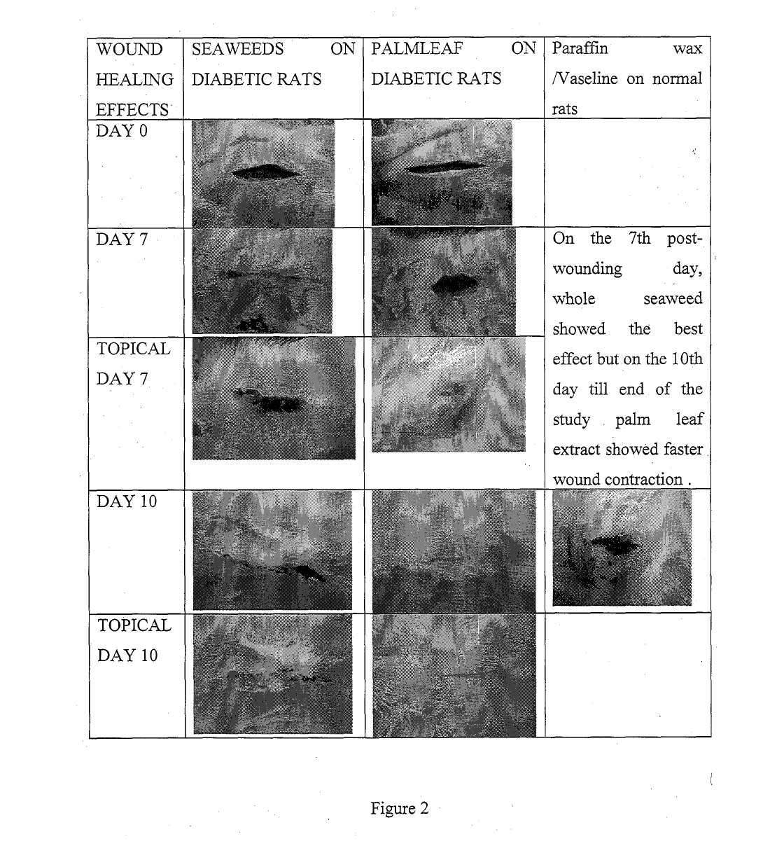

### DETAILED DESCRIPTION OF THE INVENTION

It is to be understood that the present invention may be embodied in other specific forms and is not limited to the sole embodiment described above. However modification and equivalents of the disclosed concepts such as those which readily occur to one skilled in the art are intended to be included within the scope of the claims which are appended thereto.

The term “pharmaceutically effective amount” used herein throughout the specification refers to the amount of the active ingredient, the extract, to be administered orally to the subject to trigger the desired effect without or causing minimal toxic adverse effect against the subject. One skilled in the art should know that the effective amount can vary from one individual to another due to the external factors such as age, sex, diseased state, races, body weight, formulation of the extract, availability of other active ingredients in the formulation and so on.

With respect to the preferred embodiment of the present invention, a method for preparing extracts from plants of the Ipomoea, Arecaceae family and seaweeds is disclosed. The method basically comprises the steps of pre-treating parts from plants of Ipomoea, Arecaceae family and/or seaweeds; extracting the pre-treated parts using a alcoholic solvent with or without combination with a co-solvent, which may be aqueous or organic; removing the pre-treated parts from the used polar or non-polar solvent; and concentrating the used solvent to acquire the herbal extract. The disclosed method is found to be effective in isolating the favoured compounds, peptides or active metabolites which provides a wound healing enhancing effects or health promoting effect towards the wounded area upon administrating an effective amount of the disclosed extract orally or topically towards the subject.

According to another preferred embodiment of the disclosed method, the vegetative parts are pre-treated before subjecting to extraction. The pre-treatment process may involve a washing step to clean any dirt or physical pollutants captured at the surface of the vegetative parts. Other pre-treatment step is preferably reducing the moisture content of the vegetative parts by any known means or approaches in the art to improve the extraction rate and yield. In the preferred embodiment, the vegetative parts are subjected to drying in an oven at 30-100° C. The drying temperature shall not be set too high as such practice may degrade the active metabolites and compounds contained within the vegetative parts. The preferred temperature shall range from 40° C. to 70° C. Other pre-treatment steps that can be employed are preparing the dried vegetative parts into small fragments, or pulverization to paste or powdery form prior to the soaking or solvent extraction step. The fragmentised portion or pulverized vegetative parts can greatly increase the available contact surface of the dried vegetative parts that are exposed to extraction solvent thus enhance the rate as well as the yield of the extraction method. Possibly, the pre-treated vegetative parts may be subjected to a plurality occasion of extraction using different types of extraction solvent to obtain the optimal yield.

Though mere soaking the pre-treated vegetative parts into the extraction alcoholic solvent with or without combination with a co-solvent, which may be aqueous or organic shall enable the extraction of the active compounds due to the polarity attraction of the extraction solvent, the process may be accelerated by stirring the extraction mixture, or the use of heat and pressure, both of the extraction solvent and the pre-treated vegetative parts, during the time the extraction is conducted. It is important to note that the extracted compounds from the vegetative parts are mainly constituted of phenolic compounds, pigments, proteins, minerals, polysaccharides, lipids, small peptides and other bioactive compounds. Moreover, applying appropriate amount of heat energy to the extraction system is another feasible approach to enhance the extraction rate and yield. Relying upon the solvent utilized, the heating is most preferred within the range of 30° C. to 100° C. Precaution should be taken into consideration that denaturalisation possibly occurs at high temperature of heating.

It is important to note that the vegetative parts (especially foliages) used in the disclosed method in the preferred embodiment derives from the plant species of, but not limited to, Ipomoea batatas, Euchema cottonii, Kappaphycus alvarezii, Cauleipa lentillifera and Sargassum polycystum, Elaeis guineensis, Elaeis oleifera, Phoenix dactylifera and Cocos nucifera. The extracts obtained from the abovementioned plant species are found acceptable in both taste and fragrance thus render them suitable to be incorporated into edible products or as capsules and tablets.

Pursuant to further embodiment of the disclosed method, the extraction solvent preferably employed is, but not limited to, any one or combination of alcohol, water, acetone, acetonitrile, chloroform, dichloromethane and petroleum ether. The desired compounds to be extracted from the vegetative parts are mainly constituted of, but not limited to, proteins, biophenols, lipids, saccharides, minerals and small peptides. Due to polarity of these compounds, the solvent is found to be effective in extracting these desired compounds from the plant matrix. To acquire optimal yield by using effective amount of extraction solvent, the ratio of the pre-treated vegetative parts to the extraction solvent is preferably 1-3: 6-10 (w/v).

Preferably, the pre-treated vegetative parts after subjecting to extraction can be separated by any known means and approaches in the art for disposal. Vacuum filtration is most preferred as such approach is commonly available. Similarly, vaporization of the used extraction solvent to concentrate compounds can be performed by different approaches. For, example drying the used extraction solvent using heat energy or vacuum drying. Concentrating the compounds by dissipating the used extraction solvent shall finally reach to the stage where the compounds are concentrated to a paste or powdery form. This paste or powdery form of compounds extract can then be utilized for various applications.

Attention is now drawn to another embodiment of the present invention which involves a comestible and/or topical composition for promoting wound healing and healthcare of the cardiovascular system of an individual comprises extracts derive from vegetative parts of a plant of Ipomoea, Arecaceae and Seaweeds family using an extraction solvent. The comestibles mentioned herein can be any common daily consumed processed food such as bread, noodles, confections, chocolates, beverages, and the like. One skilled in the art shall appreciate the fact that the aforesaid extract can be incorporated into the processed comestibles, capsules, tablets or topical medicine during the course of processing. Therefore, any modification thereon shall not depart from the scope of the present invention. In more specific, the present invention is composition to be topically or orally applied for enhancing wound healing comprising an extract of vegetative parts of Ipomoea, Arecaceae plant family and/or seaweeds through extraction using alcoholic solvent with or without combination with a co-solvent, which may be aqueous or organic as an active ingredient.

As setting forth in the above description, the vegetative parts used for preparing the extract are derived from the plants of Ipomoea and/or Arecaceae plant family, optionally extract from seaweeds. More preferably, the plant is any one or combination of the plant species of, but not limited to, Ipomoea batatas, Eucheina cottonii, Kappaphycus alvarezii, Cauleipa lentillifera and Sargassum polycystuin, Elaeis guineensis, Elaeis oleifera, Phoenix dactylifera and Cocos nucifera. The inventors of the present invention discovered that the extract derives from the aforementioned species possesses both acceptable taste that confers the derived extract to be comfortably incorporated with the comestibles product, capsules, tablets or topical medicine with minimal additional refining process. Preferably, the vegetative parts employed in the present invention is foliages.

According to the preferred embodiment, the extract to be incorporated into the comestibles and medicine can be acquired from any known method not limited only to the foregoing disclosed method. Following to another embodiment, the extract is prepared in a concentrated form, preferably paste or powdery form which enables the extract to be prepared in various formulations of the comestibles, capsules, tablets or topical applications.

In line with the preferred embodiment, the extract shall be the plant metabolites which are susceptible to an extraction solvent. The compounds and small peptides with the wound healing and cardiovascular system health-promoting properties are those metabolites with or without polarity in the vegetative parts. Therefore, the extract from the vegetative parts of plants of Ipomoea, Arecaceae and Seaweeds family is preferably derives from the extraction solvent of water, alcohol, acetone, chloroform dichloromethane, petroleum ether and any combination thereof.

In view of the prominent property of promoting wound healing, tissue healing and general healthcare of the cardiovascular system by the extracts in a subject, further embodiment of the present invention includes a method comprising the step of administrating orally or topically to the subject an effective amount of an extract of vegetative parts derive from a plant of Ipomoea, Arecaceae family and/or seaweeds using an extraction polar or non-polar solvent. The Ipomoea species is preferably Ipomoea batatas, while the species of Arecaceae plant family is Elaeis guineensis, Elaeis oleifera, Phoenix dactylifera and Cocos nucifera. Optionally extraction of the seaweeds may come from any one or combination of Euchema cottonii, Kappaphycus alvarezii, Cauleipa lentillifera and Sargassum polycystum.

In accordance with the preferred embodiment, the disclosed composition of the present invention can actually be applied concurrently with other medicament to enhance wound healing, tissue healing and cardiovascular health protection by topical use of the present invention. Antibiotics such as mupirocin, metranidazole, polymyxin B, Neosporin, or bacitracin maybe applied independently or integrated into the topically applied composition of the present invention to synergistically promote wound healing in a subject.

The following example is intended to further illustrate the invention, without any intent for the invention to be limited to the specific embodiments described therein.

### Example 1

Palm, Ipomea batatas or seaweeds vegetative parts were collected, cleaned, washed and cut into small pieces and oven dried at 40° C. overnight. The dried material was ground using a blender and extracted three times with hot and cold alcohol (1:10 v/v) and three times with hot and cold water or with mixtures of chloroform and alcohol. Other solvent such as acetone may be used as a medium for the extraction. This is a process designed to separate soluble compound by diffusion from a solid matrix (plant tissue) using a liquid matrix (solvent). Alcohol, water, chloroform and acetone has produced good yield in extracting the active components. The extraction was done a few times. The pooled extracts were vacuum-dried at 40° C. and stored until used.

### Example 2

Enhanced wound healing, anti-diabetic, anti-oxidative, antimicrobial and organ protective properties evaluation had been conducted on the different products from the different source of Palm, Ipomea batatas or seaweeds vegetative parts.

The wounding procedure was carried out using ketamine (1 ml/kg body weight) anesthetized normal or diabetic Sprague Drawley rats in different wound models (Incision and excision). The extract was given daily to the rats orally in the case of incision and excision wound for 15 days, or till the day of epithelization.

For Incision Wounds, 2 cm incisions were made through the full thickness of the skin. Wound contraction was measured daily until the 10th post-wounding day.

For Excision Wound, a circular piece of full thickness (˜20 mm diameter) was cut off from an area on the neck of the rat. Wounds were traced on 1 mm² graph paper on the lay of wounding and subsequently until healing was complete. Changes in wound area were calculated, for rate of wound contraction. Number of days required for falling of eschar without any residual raw wound indicate the period of epithelization.

Results of the test are shown in both Table 1, Table 2 and Table 3.

TABLE 1
The excision wound area (mm²) of different groups over a period of 15 days

| Group | Day 0 | Day 3 | Day 6 | Day 9 | Day 12 | Day 15 |
|---|---|---|---|---|---|---|
| EO1 | 259.3 ± 25.4 | 237.0 ± 23.4 | 160.5 ± 26.5 | 51.5 ± 5.61 | 24.5 ± 13.9 | 0.0 ± 0.0 |
| EO2 | 241.0 ± 17.0 | 219.0 ± 15.3 | 144.0 ± 9.2 | 58.8 ± 12.5 | 9.04 ± 4.78 | 0.0 ± 0.0 |
| EW1 | 220.0 ± 9.54 | 212.5 ± 8.98 | 175.5 ± 10.1 | 110.5 ± 17.8 | 67.5 ± 11.6 | 37.47 ± 4.85 |
| EW2 | 259.0 ± 15.3 | 248.5 ± 16.1 | 189.0 ± 17.0 | 85.0 ± 15.3 | 62.47 ± 8.78 | 32.5 ± 8.23 |
| Whole | 273.3 ± 25.1 | 271.0 ± 27.5 | 149.8 ± 15.6 | 93.5 ± 18.2 | 73.0 ± 25.1 | 70.8 ± 24.6 |
| Honey (+ctrl) | 261.5 ± 33.8 | 242.5 ± 35.7 | 159.0 ± 4.84 | 102.0 ± 10.7 | 58.0 ± 19.2 | 13.33 ± 7.07 |
| Water (−ctrl) | 269.0 ± 27.2 | 267.5 ± 27.4 | 198.0 ± 18.5 | 166.7 ± 14.3 | 144.0 ± 18.7 | 115.7 ± 12.6 |
| I. batatas | 268.0 ± 26.1 | 226.0 ± 24.3 | 159.4 ± 25.4 | 51.5 ± 5.61 | 24.3 ± 12.8 | 0.0 ± 0.0 |

value are mean ± SE of six animals in each group
EO1: Seaweeds Cold alcoholic extract;
EO2: Seaweeds Hot alcoholic extract;
EW1: Seaweeds Cold water extract:
EW2: Seaweeds Hot water extract
I. batatas: Ipomoea batatas extract

TABLE 2
Excision Wound contraction (%) of different groups over a period of 15 days

| | Healing % | Healing % | Healing % | Healing % | Healing % |
|---|---|---|---|---|---|
| Group | at 3rd day | at 6th day | at 9th day | at 12th day | at 15th day |
| EO1 | 8.67 ± 0.42 | 37.92 ± 8.61 | 79.96 ± 1.57 | 92.35 ± 4.00 | 100 ± 0.00 |
| EO2 | 9.08 ± 0.51* | 36.35 ± 9.83 | 75.95 ± 5.93 | 96.04 ± 2.07 | 100 ± 0.00 |
| EW1 | 3.36 ± 0.78 | 19.82 ± 5.04 | 50.09 ± 6.83 | 70.16 ± 4.97 | 83.44 ± 2.01 |
| EW2 | 4.20 ± 0.71 | 27.48 ± 3.13 | 67.42 ± 4.98 | 77.59 ± 2.69 | 88.20 ± 3.23 |
| Whole | 1.23 ± 0.98 | 43.81 ± 6.44 | 64.21 ± 7.37 | 66.9 ± 12.8 | 67.90 ± 12.5 |
| Honey (+ctrl) | 8.23 ± 2.27 | 34.08 ± 8.50 | 58.34 ± 6.53 | 72.77 ± 9.79 | 93.76 ± 3.25 |
| Water (−ctrl) | 0.63 ± 0.40 | 23.48 ± 8.81 | 35.40 ± 7.26 | 40.30 ± 13.00 | 52.66 ± 8.56 |

In the excision wound model, the percent of wound contraction was measured daily. The percentage wound contraction was nearly 100% for both the doses of alcoholic extracts in 15 days while for the control it was 22 days (P<0.05). The percentage wound contraction was nearly 100% for the both the doses of aqueous extract in 16 days in comparison with controls (P<0.05). In the above physiological and pharmacological parameters of wound healing, ethanol extract of seaweeds and leaves of Ipomoea batatas was more effective than the aqueous extract.

TABLE 3
Incision Wound contraction (%) of normal and diabetic rats
given seaweeds or palm leaf extract over a period of 10 days
Wound contraction

| Group | Day 0 | Day 4 | Day 7 | Day 10 |
|---|---|---|---|---|
| NC | 20.00 ± 0.00 | 15.96 ± 1.56 | 6.01 ± 1.44 | 0.74 ± 0.54 |
| NS | 20.00 ± 0.00 | 14.73 ± 2.28 | 9.91 ± 3.41 | 5.84 ± 2.38 |
| NP | 21.25 ± 1.25 | 19.21 ± 1.73 | 13.15 ± 1.17 | 6.16 ± 4.37 |
| DC | 20.00 ± 0.00 | 20.75 ± 2.24 | 20.74 ± 2.23 | 13.88 ± 0.52 |
| DS | 23.33 ± 1.67 | 21.11 ± 1.95 | 18.60 ± 1.34 | 12.54 ± 6.82 |
| DP | 20.00 ± 0.00 | 16.27 ± 0.99 | 15.34 ± 1.06 | 11.54 ± 1.83 |
| DTS | 20.00 ± 0.00 | 17.71 ± 0.68 | 15.13 ± 0.19 | 13.09 ± 1.81 |
| DTP | 21.25 ± 1.25 | 18.80 ± 1.20 | 13.70 ± 1.18 | 8.63 ± 2.90 |

Normal (NC), Normal Seaweed (NS), Normal Oil Palm leaf (NP), Control diabetic (DC), Diabetic seaweed (DS), Diabetic oil palm leaf (DP), Diabetic topically seaweed (DTS), Diabetic topically oil palm leaf (DTP)
Mean ± SE

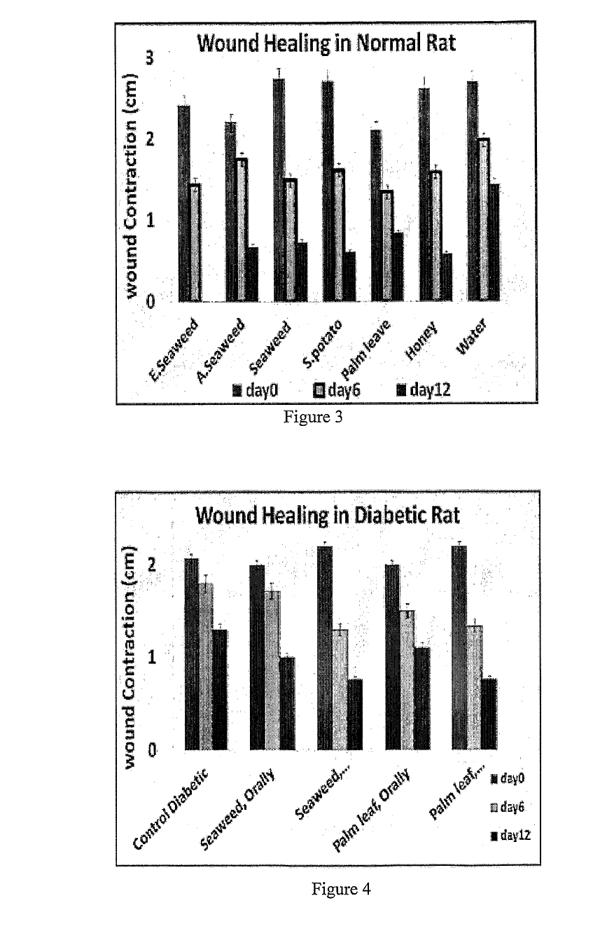

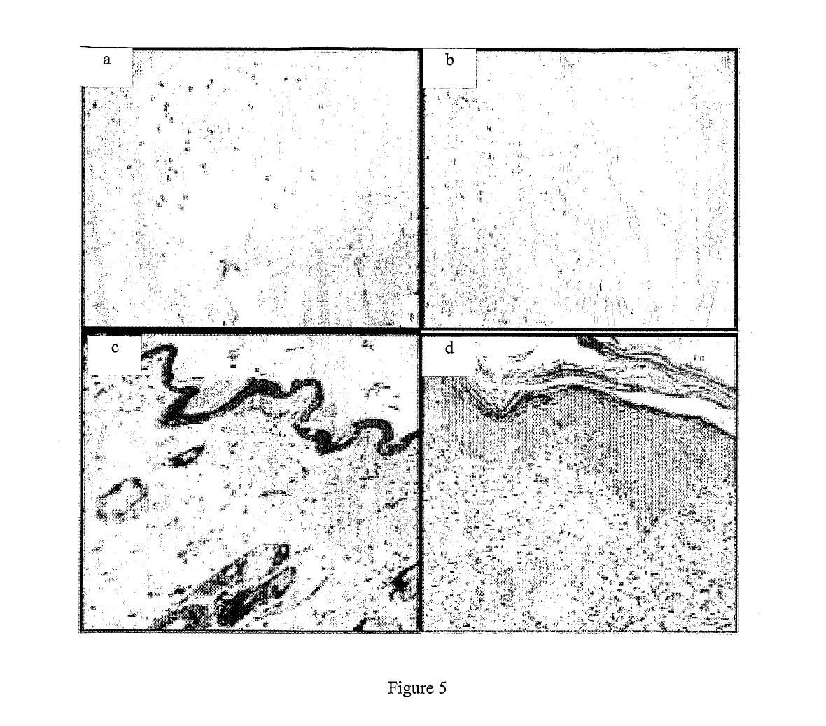

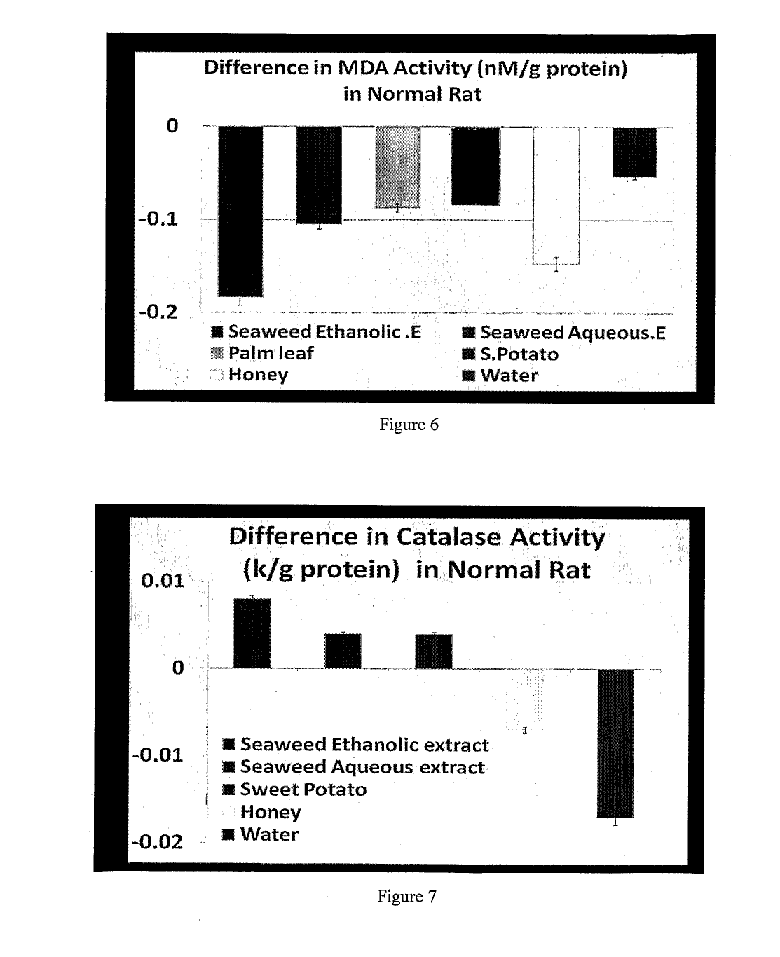

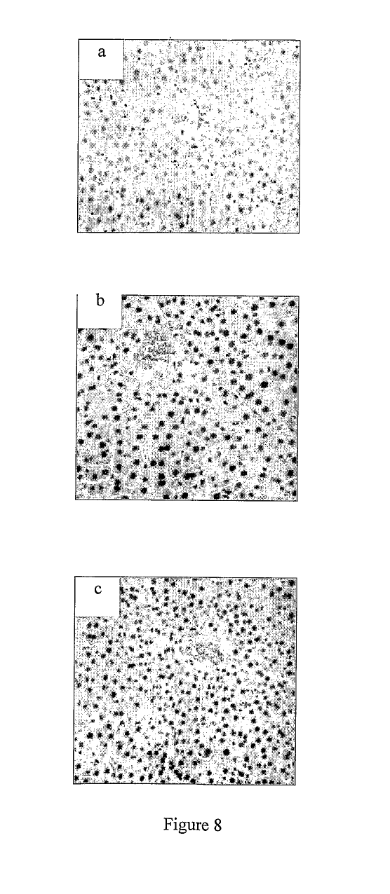

The many stages of wound healing (granulation, collagen and scar formation) run simultaneously, but without help from each other. These extracts showed accelerated healing in both incision and excision wounds compared with untreated groups. The increased wound contraction rate may be caused by stimulation of inflammatory α-chemokine interleukin-8, that help recruit various inflammatory cells, fibroblasts and keratinocytes, and augment the gap junctional intracellular communication in cultured fibroblasts to accelerate the maturation of granulation tissue as shown in the FIGS. 3 to 6.

### Example 3

10×9 male STZ-induced diabetic Sprague-dawley rats of 200-250 g (8 weeks old) (D) were used for carrying out the experiment where DC: Untreated diabetic rats (D) as negative control; GB: D+3 mg/kg glibenclamide (drug+ve control); SW (50-100 mg/kg): D+(50-100)mg/kg seaweeds extract; OPL (50-200 mg/kg): D+(50-200)mg/kg oil palm leaves extract; NF: Normal rats as positive control and Diabetes were induced by injecting 60 mg/kg streptozotocin (STZ). Diabetes:Fasting blood glucose >8 mmol/L˜5 days after STZ induction.

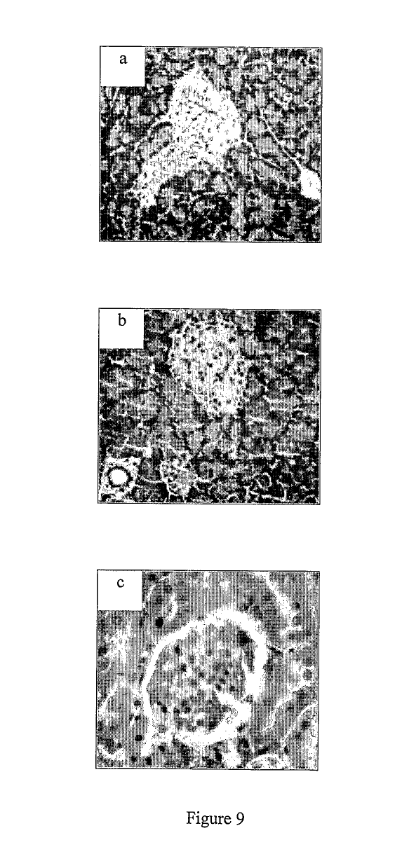

The histology studies on liver and kidney of the untreated diabetic rats, diabetic rats treated with seaweed and palm extracts are shown in FIGS. 8 and 9 respectively.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1-6. (canceled)

7. A composition for enhancing skin wound healing, comprising:
an extract of plant parts of the Arecaceae plant family, wherein the extract is acquired using a polar solvent.

8. The composition according to claim 7, wherein the plant part is foliages.

9. The composition according to claim 7, wherein the composition is topically applied.

10. The composition according to claim 7, wherein the composition is orally administered.

11. The composition according to claim 7, wherein the Arecaceae plant is selected from the group consisting of Elaeis guineensis, Elaeis oleifera, Phoenix dactylifera, Cocos nucifera, and combinations thereof.

12. The composition according to claim 7, further comprising seaweed selected from the group consisting of Eucheuma cottonii, Kappaphycus alvarezii, Caulerpa lentillifera, Sargassum polycystum, and combinations thereof.

13. The composition according to claim 7, wherein the polar solvent is selected from the group consisting of alcohol, water, acetone, acetonitrile, dichloromethane, and combinations thereof.

14. A composition to be orally administered for enhancing skin wound healing, comprising:
an extract of foliage of Ipomoea batatas, wherein the extract is acquired using a polar solvent selected from the group consisting of alcohol, water, acetone, acetonitrile, dichloromethane, and combination thereof.
